(19) United States
(12) Patent Application Publication
(10) Pub. No.: US 2026/0018276 A1
(43) Pub. Date: Jan. 15, 2026

(54) VENDOR NEUTRAL ARTIFICIAL INTELLIGENCE INFUSED PROTOCOL CREATION AND OPTIMIZATION

(71) Applicant: GE Precision Healthcare LLC, Waukesha, WI (US)

(72) Inventors: Manpreet Singh, Bangalore (IN); Angelo Fernandes, Bangalore (IN); Amey Walke, Bangalore (IN)

(21) Appl. No.: 18/772,453

(22) Filed: Jul. 15, 2024

(51) Int. Cl. G16H 30/40; G16H 40/60; G16H 50/20

(52) U.S. Cl. CPC G16H 30/40; G16H 40/60; G16H 50/20

(57) ABSTRACT

A computer-implemented method for optimizing protocols for medical imaging scanners includes receiving, at a processing system including one or more processors, a planned protocol from an organization for a medical imaging scanner. The computer-implemented method also includes utilizing, via the processing system, an artificial intelligence-based algorithm to generate suggested protocol parameters to optimize the planned protocol based at least on the planned protocol. The computer-implemented method further includes outputting, via the processing system, from the artificial intelligence-based algorithm an optimized protocol for the planned protocol based on one or more of the suggested protocol parameters. The computer-implemented method includes modifying, via the processing system, settings of the medical imaging scanner when the optimized protocol is utilized for a scan of a subject with the medical imaging scanner. The computer-implemented method includes executing, via the processing system, a scan of the subject with the medical imaging scanner utilizing the optimized protocol.

### BACKGROUND

The subject matter disclosed herein relates to vendor neutral artificial intelligence infused protocol creation and optimization.

Imaging scanners used protocols to scan patients. Many hospital organizations maintain their own sets of protocols to be utilized for specific scenarios and operations. However, these protocols need to be maintained for each scanner model as they are incompatible across vendors (e.g., original equipment manufacturers) and are often incompatible across the same scanner model family. Protocol compatibility can be defined as the ability to use the protocols from one scanner to do the scan in another scanner to achieve similar results without the need of manual modifications to the protocols. Individual modifications are not considered as it can depend on the preference of the person prescribing the scan. Creating a new protocol outside the scanner can be a challenge without having the scanner protocol management software in place. Likewise, driving a common outcome across the protocols is a challenge as it cannot be done outside the protocol management software. Thus, there is a need to cross transfer the protocols across various vendors to have consistency in the radiology department for which there is no solution presently in the field.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for optimizing protocols for medical imaging scanners is provided. The computer-implemented method includes receiving, at a processing system including one or more processors, a planned protocol from an organization for a medical imaging scanner. The computer-implemented method also includes utilizing, via the processing system, an artificial intelligence-based algorithm to generate suggested protocol parameters to optimize the planned protocol based at least on the planned protocol. The computer-implemented method further includes outputting, via the processing system, from the artificial intelligence-based algorithm an optimized protocol for the planned protocol based on one or more of the suggested protocol parameters. The computer-implemented method even further includes modifying, via the processing system, settings of the medical imaging scanner when the optimized protocol is utilized for a scan of a subject with the medical imaging scanner. The computer-implemented method still further includes executing, via the processing system, a scan of the subject with the medical imaging scanner utilizing the optimized protocol.

In another embodiment, a computer-implemented method for optimizing protocols for medical imaging scanners is provided. The computer-implemented method includes receiving, at a processing system including one or more processors, clinical requirements for a scan using a medical imaging scanner of an organization. The computer-implemented method also includes receiving, at the processing system, information specific to hardware and software of the medical imaging scanner. The computer-implemented method further includes receiving, at the processing system, user input of one or more desired outcomes for the scan. The computer-implemented method even further includes utilizing, via the processing system, a generative artificial intelligence-based model to generate a protocol for performing the scan using the medical imaging scanner based on the clinical requirements and the one or more desired outcomes for the scan.

In a further embodiment, a computer-implemented method for optimizing protocols for medical imaging scanners is provided. The computer-implemented method includes receiving, at a processing system including one or more processors, existing planned protocols from an organization for performing a scan with a first medical imaging scanner. The computer-implemented method also includes receiving, at the processing system, information specific to hardware and software of the first medical imaging scanner. The computer-implemented method further includes receiving, at the processing system, additional information specific to hardware and software of a second medical imaging scanner different from the first medical imaging scanner, wherein the second medical imaging scanner is of a different manufacturer and/or a different model from the first medical imaging scanner. The computer-implemented method even further includes receiving, at the processing system, user input of one or more desired outcomes for a respective scan with the second medical imaging scanner. The computer-implemented method still further includes utilizing, via the processing system, a generative artificial intelligence-based model to generate a protocol for performing the respective scan using the second imaging scanner based on the existing planned protocols and the one or more desired outcomes for the respective scan.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 is a schematic diagram of a system configured to optimize and/or create protocols for medical imaging scanners, in accordance with aspects of the present disclosure;

FIG. 2 is a flow chart of a method for optimizing protocols for medical imaging scanners (e.g., individual protocol optimization), in accordance with aspects of the present disclosure;

FIG. 3 is a flow chart of a process for optimizing protocols for medical imaging scanners (e.g., individual protocol optimization), in accordance with aspects of the present disclosure;

FIG. 4 is a flow chart of a method for optimizing protocols for medical imaging scanners (e.g., protocol set optimization), in accordance with aspects of the present disclosure;

FIG. 5 is a flow chart of a process for optimizing protocols for medical imaging scanners (e.g., protocol set optimization), in accordance with aspects of the present disclosure;

FIG. 6 is a flow chart of a method for optimizing protocols for medical imaging scanners (e.g., via propagation), in accordance with aspects of the present disclosure;

FIG. 7 is a flow chart of a process for optimizing protocols for medical imaging scanners (e.g., via propagation), in accordance with aspects of the present disclosure;

FIG. 8 is a flow chart of a method for optimizing protocols for medical imaging scanners (e.g., individual protocol optimization based on past performed scans), in accordance with aspects of the present disclosure;

FIG. 9 is a flow chart of a process for optimizing protocols for medical imaging scanners (e.g., individual protocol optimization based on past performed scans), in accordance with aspects of the present disclosure;

FIG. 10 is a flow chart of a method for optimizing protocols for medical imaging scanners (e.g., protocol set optimization based on past performed scans), in accordance with aspects of the present disclosure;

FIG. 11 is a flow chart of a process for optimizing protocols for medical imaging scanners (e.g., protocol set optimization based on past performed scans), in accordance with aspects of the present disclosure;

FIG. 12 is a flow chart of a process for generating a generative artificial intelligence model for use in in creating/optimizing protocols, in accordance with aspects of the present disclosure;

FIG. 13 is a flow chart of a process for refining a generative artificial intelligence model for use in in creating/optimizing protocols, in accordance with aspects of the present disclosure;

FIG. 14 is a flow chart of a method for creating new protocols for medical imaging scanners, in accordance with aspects of the present disclosure;

FIG. 15 is a flow chart of a process for creating new protocols for medical imaging scanners, in accordance with aspects of the present disclosure;

FIG. 16 is a flow chart of a method for translating protocols for medical imaging scanners across scanner models and vendors, in accordance with aspects of the present disclosure; and

FIG. 17 is a flow chart of a process for translating protocols for medical imaging scanners across scanner models and vendors, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

The term processor, processing system, or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

As used herein, the term “computing system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the terms “application”, “application module” (or “module”), “engine”, or “program”, or “plugin” refers to one or more sets of computer software instructions (e.g., computer programs and/or scripts) executable by one or more processors of a computing system to provide particular functionality. Computer software instructions can be written in any suitable programming languages, such as C, C++, C#, Pascal, Fortran, Perl, MATLAB, SAS, SPSS, JavaScript, AJAX, and JAVA. Such computer software instructions can comprise an independent application with data input and data display aspects (e.g., modules). Alternatively, the disclosed computer software instructions can be classes that are instantiated as distributed objects. The disclosed computer software instructions can also be component software, for example JAVABEANS or ENTERPRISE JAVABEANS. Additionally, the disclosed applications or engines can be implemented in computer software, computer hardware, or a combination thereof.

As used herein, the terms “automatic” and “automatically” refer to actions that are performed by a computing device or computing system (e.g., of one or more computing devices) without human intervention. For example, automatically performed functions may be performed by computing devices or systems based solely on data stored on and/or received by the computing devices or systems despite the fact that no human users have prompted the computing devices or systems to perform such functions. As but one non-limiting example, the computing devices or systems may make decisions and/or initiate other functions based solely on the decisions made by the computing devices or systems, regardless of any other inputs relating to the decisions.

Deep learning (DL) approaches discussed herein may be based on artificial neural networks, and may therefore encompass one or more of deep neural networks, fully connected networks, convolutional neural networks (CNNs), transformer-based networks, unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides for systems and methods for optimizing an organization's (e.g., hospital organization or any other health care providing organization) scanning protocols (e.g., radiology protocols) for medical imaging scanners. The medical imaging scanners may be part of a computed tomography imaging system, a digital radiography system, an ultrasound imaging system, a magnetic resonance imaging system, a nuclear medicine imaging system, or any other type of medical imaging system. In certain embodiments, the systems and methods enable an organization to optimize (i.e., to make best or most effective use of or improve (e.g., in image quality or other imaging factor) over the prior protocol(s)) its own protocol set with the use of a proprietary algorithm and artificial intelligence techniques to remove the protocols giving subpar scan results and boost the quality of other protocols based on suggestions from the algorithm. In certain embodiments, the systems and methods utilize generative artificial intelligence to help the organization create new protocols similar to their own protocol set based on clinical scenario submitted by a user. The generative artificial intelligence may be utilized to optimize protocols not just within the scanner model but also across the model family and different vendors. In certain embodiments, the systems and methods leverage an organization specific radiology large language model (ORaLLM) to create/translate protocols and vendors (e.g., original equipment manufacturers) which eliminates the need to manually create protocols from scratch whenever a hospital organization buys a new imaging device from a different vendor than the one they currently have. The artificial intelligence utilized in the disclosed embodiments is vendor neutral.

The disclosed systems and methods enable hospitals to improve efficiency and to obtain a better technical edge with their optimized planned protocol set. Also, the disclosed systems and methods enable hospitals to maintain fewer or smaller protocol sets as duplicated protocols yielding the same scan outcome are eliminated. Further, the disclosed systems and methods enable hospitals to save time in creating new protocols with the help of generative artificial intelligence. Even further, the newly created protocols (created via artificial intelligence) will require less manual tweaking since they were fine-tuned on an organization's own protocol set to match each organization's preferences and tastes. Still further, the disclosed systems and methods enable hospitals to save time and money by translating their own optimized protocol set to a different scanner across models and vendors instead of creating it from scratch. The disclosed systems and methods provide better scan results for patients which results in a faster diagnosis due to optimized protocols by hospitals. The disclosed systems and methods enable the same scan results for a patient for a given prescription across various vendor scanners.

In certain embodiments, an artificial intelligence-based algorithm takes an organization's planned protocol set and optimizes them based on protocol parameters suggested by the algorithm. In certain embodiments, the user can additionally train the algorithm for customized results. In certain embodiments, another artificial intelligence-based algorithm may identify improvements done to a planned protocol and propagate these improvements to other planned protocols. In certain embodiments, another artificial intelligence-based algorithm may optimize an organization's planned protocol sets based on desired scan outcome(s) (e.g., reduced dose, better image quality, etc.) desired by the user and considering the past scans performed on the scanner.

In certain embodiments, a generative artificial intelligence model is pre-trained with original equipment manufacturer data (e.g., from different vendors) which can be further fine-tuned based on the organization's own protocols without exposing it outside the organization. In certain embodiments, a generative artificial intelligence model may be utilized to create new protocols across various scanner models and vendor based on clinical requirements and desired scan outcome(s) submitted by the user. In certain embodiments, a generative artificial intelligence model may be utilized to translate an organization's existing planned protocol set form one scanner model to other models and across various vendors. The disclosed embodiments maintain compliance with healthcare privacy and security standards across geographical regions.

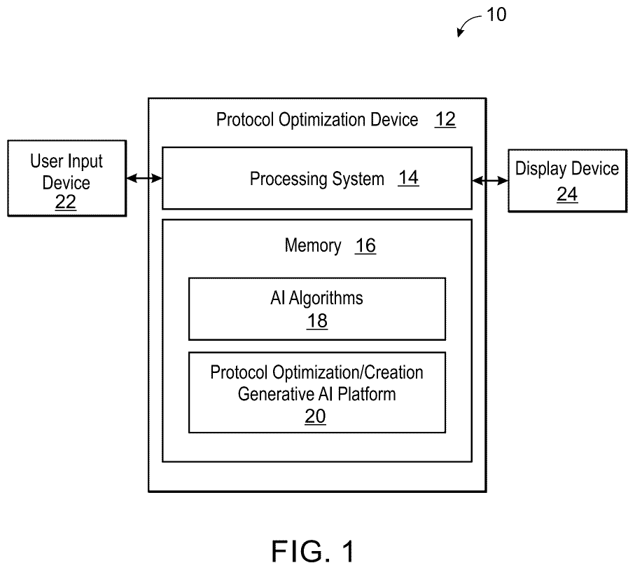

FIG. 1 is a schematic diagram of a system 10 (e.g., protocol optimization/creation system) configured to optimize and/or create protocols (e.g., scanning or radiological protocols) for medical imaging scanners. A scanning protocol takes into the account the imaging modality, the purpose of the scan, the anatomical region of interest to be images, and scanning parameters (e.g., acquisition parameters). As depicted, the system 10 includes a protocol optimization device 12 (e.g., implemented in a computing device). The protocol optimization device 12 may be located on a medical imaging system or may located remotely from any medical imaging system. The protocol optimization device 12 is configured to optimize or create protocols for medical imaging scanners that belong to an organization such as a hospital organization. The protocol optimization device 12 is configured to utilize one or more artificial intelligence-based algorithms and/or protocol optimization/creation agent framework, via generative artificial intelligence based reasoning, to optimize and/or create protocols for use with the medical imaging scanners of an organization. The artificial intelligence utilized by the protocol optimization device is vendor neutral.

The protocol optimization device 12 includes one or more processors forming a processing system 14 configured to execute machine readable instructions stored in non-transitory memory 16. A processor of the processing system 14 may be single core or multi-core, and the programs executed thereon may be configured for parallel or distributed processing. In some embodiments, the processing system 14 may optionally include individual components that are distributed throughout two or more devices, which may be remotely located and/or configured for coordinated processing. In some embodiments, one or more aspects of the processing system 14 may be virtualized and executed by remotely-accessible networked computing devices configured in a cloud computing configuration.

The protocol optimization device 12 also includes the non-transitory memory 16. The non-transitory memory 16 may store one or more artificial intelligence (AI)-based algorithms 18. In certain embodiments, the artificial intelligence-based algorithms 18 are configured to take an organization's existing planned protocols (e.g., individually or as a set) and to optimize them based on protocol parameter suggested by the algorithms 18. In certain embodiments, the artificial intelligence-based algorithms 18 are configured to identify improvements done to a planned protocol and to propagate these improvements to other planned protocols. In certain embodiments, the artificial intelligence-based algorithms 18 are configured to optimize the organization's planned protocol set based on the desired scan outcome(s) (e.g., reduced dose, better image quality, etc.) desired by the user and considering past scans performed on the scanner.

The non-transitory memory 16 may store a protocol optimization/creation generative AI platform 20. In certain embodiments, the protocol optimization/creation generative AI platform 20 includes an agent framework and a large language model. The agent framework and the large language model are configured to act together to serve as the main controller that controls a flow of operations to complete a task or user request. The agent framework is a set of predefined functions compiled into the agent for each resource type. The large language model is a very large deep learning model pre-trained on vast amounts of data. The underlying transformer is a set of neural networks that consist of an encoder and a decoder with self-attention capabilities. The encoder and decoder extract meanings from a sequence of text and understand the relationships between words and phrases in it. The large language model can be, in some examples, open sourced.

In certain embodiments, the protocol optimization/creation generative AI platform 20 includes a generative AI model pre-trained with original equipment manufacturer (OEM) data (e.g., vendor data) that is further fine-tuned based on the organization's own protocols without exposing outside the organization. In certain embodiments, the protocol optimization/creation generative AI platform 20 is configured to create new protocols across various scanner models and vendors based on clinical requirements and desired scan outcome(s) submitted by the user. In certain embodiments, the protocol optimization/creation generative AI platform 20 is configured to translate an organization's existing planned protocol sets from one scanner model to another scanner model across various vendors.

In some embodiments, non-transitory memory 16 may include components disposed at two or more devices, which may be remotely located and/or configured for coordinated processing. In some embodiments, one or more aspects of non-transitory memory 16 may include remotely-accessible networked storage devices configured in a cloud computing configuration.

User input device 22 may include one or more of a touchscreen, a keyboard, a mouse, a trackpad, a motion sensing camera, or other device configured to enable a user to interact with the protocol optimization device 12. In one example, user input device 22 may enable a user to provide preferences for a scan (e.g., a desired scan outcome, optimization criteria, etc.), acceptance/rejection of a suggestion/protocol provided by the protocol optimization device 12, and/or a user prompt to create new protocol(s) and/or translate protocol(s).

Display device 24 may include one or more display devices utilizing virtually any type of technology. In some embodiments, the display device 24 may include a computer monitor, and may display suggestions related to the optimization of protocols or other information. Display device 24 may be combined with the processing system 14, the non-transitory memory 16, and/or the user input device 22 in a shared enclosure, or may be peripheral display devices and may comprise a monitor, touchscreen, projector, or other display device known in the art, which may enable a user to view data (e.g. suggestions) and/or interact with various data stored in the non-transitory memory 16.

The processing system 14 is configured to receive a planned protocol from an organization for a medical imaging scanner. The processing system 14 is also configured to utilize an artificial intelligence-based algorithm to generate suggested protocol parameters to optimize the planned protocol based at least on the planned protocol. The processing system 14 is also configured to output from the artificial intelligence-based algorithm an optimized protocol for the planned protocol based on one or more of the suggested protocol parameters. The processing system 14 is also configured to modify settings of the medical imaging scanner when the optimized protocol is utilized for a scan of a subject with the medical imaging scanner. The processing system 14 is also configured to execute a scan of the subject with the medical imaging scanner utilizing the optimized protocol.

In certain embodiments, the processing system 14 is configured to receive user input of desired optimization criteria. In certain embodiments, the processing system 14 is also configured to receive information specific to hardware and software of the medical imaging scanner. In certain embodiments, the processing system is further configured to utilize the artificial intelligence-based algorithm to generate suggested protocol parameters to optimize the planned protocol based on the planned protocol, the desired optimization criteria, and the information specific to the hardware and the software of the medical imaging scanner.

In certain embodiments, the processing system 14 is configured to receive additional user input of acceptance of one or more of the suggested protocol parameters and/or rejection of one or more of the suggested protocol parameters. In certain embodiments, the processing system is also configured to outputting from the artificial intelligence-based algorithm the optimized protocol for the planned protocol based on the one or more of the suggested protocol parameters accepted via the additional user input. In certain embodiments, the processing system 14 is configured to train the artificial intelligence-based algorithm based on the one or more of the suggested protocol parameters accepted via the additional user input.

In certain embodiments, the processing system 14 is configured to receive a plurality of planned protocols from the organization for the medical imaging scanner. In certain embodiments, the processing system 14 is also configured to receive respective scan outcomes for each planned protocol of the plurality of planned protocols. In certain embodiments, the processing system 14 is further configured to utilize the artificial intelligence-based algorithm to group the plurality of planned protocols into different protocol sets based on the respective scan outcomes. In certain embodiments, the processing system 14 is even further configured to output from the artificial intelligence-based algorithm the different protocol sets. In certain embodiments, the processing system 14 is yet further configured to receive user input of acceptance of one or more planned protocols within each protocol set of the different protocol sets and/or rejection of one or more of the planned protocols within each protocol set of the different protocol sets. In certain embodiments, the processing system 14 is further configured to output from the artificial intelligence-based algorithm optimized protocol sets for the plurality of planned protocols based on the one or more planned protocols within each protocol set of the different protocol sets accepted via the user input.

In certain embodiments, the processing system 14 is configured to utilize the artificial intelligence-based algorithm to apply changes to all other planned protocols from the organization for the medical imaging scanner based on respective changes to the planned protocol to generate the optimized protocol. In certain embodiments, the processing system 14 is also configured to output from the artificial intelligence-based algorithm the other planned protocols with applied changes. In certain embodiments, the processing system 14 is further configured to receiving user input of acceptance of one or more of the other planned protocols with the applied changes and/or rejection of one or more of the other planned protocols with the applied changes. In certain embodiments, the processing system 14 is even further configured to output from the artificial intelligence-based algorithm respective improved protocols for the other planned protocols where the applied changes are accepted via the user input.

In certain embodiments, the processing system 14 is configured to receive a plurality of performed protocols for the planned protocol, wherein each performed protocol of the plurality of performed protocols was performed during a respective scan with respective protocol parameters. In certain embodiments, the processing system 14 is also configured to determine for each performed protocol of the plurality of performed protocols respective differences in the respective protocol parameters from protocol parameters of the planned protocol. In certain embodiments, the processing system 14 is further configured to separate the respective differences in the respective parameters into different categories. In certain embodiments, the processing system 14 is even further configured to utilize the artificial intelligence-based algorithm to generate suggested protocol parameters to optimize the planned protocol based at least on the plurality of performed protocols and the different categories.

In certain embodiments, the processing system 14 is configured to receive a plurality of performed protocols for a plurality of planned protocols from the organization for the medical imaging scanner, wherein each performed protocol of the plurality of performed protocols was performed during a respective scan with respective protocol parameters. In certain embodiments, the processing system 14 is also configured to determine for each performed protocol of the plurality of performed protocols for each respective planned protocol of the plurality of planned protocols respective differences in the respective protocol parameters from protocol parameters of the respective planned protocol. In certain embodiments, the processing system 14 is further configured to separate the respective differences in the respective parameters into different categories for the plurality of performed protocols for each respective planned protocol of the plurality of planned protocols. In certain embodiments, the processing system 14 is even further configured to utilize the artificial intelligence-based algorithm to generate suggested protocol parameters to optimize the plurality of planned protocols based at least on the plurality of performed protocols and the different categories for the plurality of planned protocols. In certain embodiments, the processing system 14 is further configured to output from the artificial intelligence-based algorithm respective planned protocols that can be improved with each suggested protocol parameter of the suggested protocol parameters. In certain embodiments, the processing system 14 is configured to receive user input of user preferences for the scan. In certain embodiments, the processing system 14 is configured to utilize the artificial intelligence-based algorithm to generate suggested protocol parameters to optimize the plurality of planned protocols based on the user preferences, the plurality of performed protocols and the different categories for the plurality of planned protocols.

The processing system 14 is configured to receive clinical requirements for a scan using a medical imaging scanner of an organization. The processing system 14 is also configured to receive information specific to hardware and software of the medical imaging scanner. The processing system 14 is further configured to receive user input of one or more desired outcomes for the scan. The processing system 14 is even further configured to utilize a generative artificial intelligence-based model to generate a protocol for performing the scan using the medical imaging scanner based on the clinical requirements and the one or more desired outcomes for the scan.

In certain embodiments, the processing system 14 is configured to receive additional user input of additional one or more desired outcomes for the scan. In certain embodiments, the processing system 14 is also configured to receive context from the generative artificial intelligence-based model. In certain embodiments, the processing system 14 is further configured to utilize the generative artificial intelligence-based model to update the protocol to generate an updated protocol based on the context and the additional user input.

The processing system 14 is configured to receive planned protocols from an organization for performing a scan with a first medical imaging scanner. The processing system 14 is also configured to receive information specific to hardware and software of the first medical imaging scanner. The processing system 14 is further configured to receive additional information specific to hardware and software of a second medical imaging scanner different from the first medical imaging scanner, wherein the second medical imaging scanner is of a different manufacturer and/or a different model from the first medical imaging scanner. The processing system 14 is even further configured to receive user input of one or more desired outcomes for a respective scan with the second medical imaging scanner. The processing system 14 is still further configured to utilize a generative artificial intelligence-based model to generate a protocol for performing the respective scan using the second imaging scanner based on the existing planned protocols and the one or more desired outcomes for the respective scan.

In certain embodiments, the generative artificial intelligence-based model includes a radiology large language model specific to the organization. In certain embodiments, the radiology large language model is fine-tuned based on protocols from the organization for the medical imaging scanner. In certain embodiments, organization specific fine-tuning of the radiology large language model is isolated from external exposure. In certain embodiments, prior to the organization specific fine-tuning, the generative artificial intelligence-based model is pre-trained based on original equipment manufacturer data for different manufacturers and different models of medical imaging scanners similar to the medical imaging scanner and the original equipment manufacturer data for the medical imaging scanner.

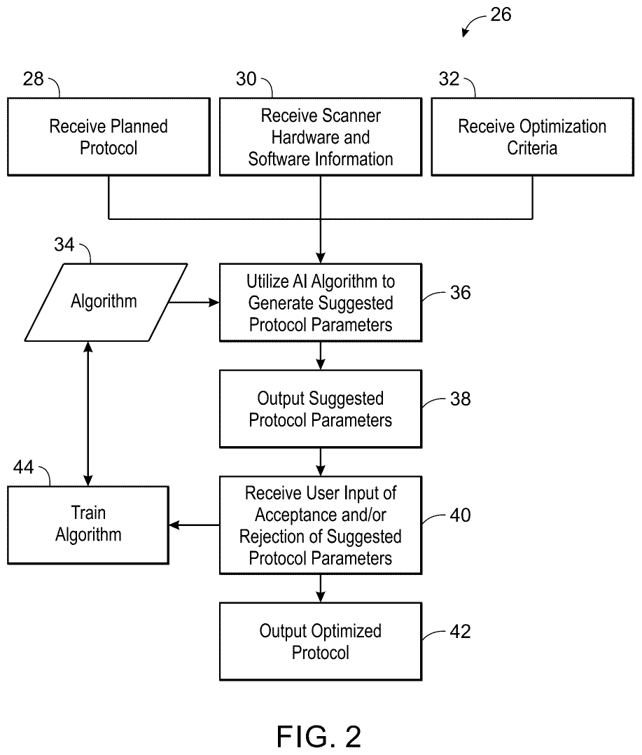

FIGS. 2-5 relate to optimization of planned protocols (e.g. individually or as a set) based on protocol parameters. FIG. 2 is a flow chart of a method 26 for optimizing protocols for medical imaging scanners (e.g., individual protocol optimization). One or more steps of the method 26 may be performed by one or more components of the protocol optimization device 12 in FIG. 1.

The method 26 includes receiving a planned protocol from an organization for a medical imaging scanner (block 28). The method 26 also includes receiving information specific to hardware and software of the medical imaging scanner (block 30). The method 26 further includes receiving user input of desired optimization criteria (block 32). Examples of a desired optimization criteria may include fixing the kilovolts (kV) at a particular level (e.g., 130 kV) and/or minimizing the dose without degrading image quality. The desired optimization criteria may vary. The method 26 also includes utilizing an artificial intelligence-based algorithm 34 to generate suggested protocol parameters to optimize the planned protocol based on the planned protocol, the information specific to the hardware and software of the medical imaging scanner, and the desired optimization criteria (block 36).

The method 26 includes outputting from the artificial intelligence-based algorithm 34 the suggested protocol parameters (e.g., display on a display having an interactive user interface) (block 38). The method 26 also includes receiving additional user input of acceptance of one or more of the suggested protocol parameters and/or rejection of one or more of the suggested protocol parameters (e.g., via the interactive user interface) (block 40). The method 26 further includes outputting from the artificial intelligence-based algorithm 34 an optimized protocol for the planned protocol based on the one or more of the suggested protocol parameters accepted via the additional user input (block 42). The optimized protocol is utilized to alter the settings of the medical imaging scanner when the optimized protocol is utilized for a scan of a subject with the medical imaging scanner. The method 26 also includes training the artificial intelligence-based algorithm 34 based on the one or more of the suggested protocol parameters accepted via the additional user input (block 44). For example, feedback learning may be utilized to train the artificial intelligence-based algorithm. In particular, reinforcement learning on human feedback (RLHF) may be utilized to match user preferences in the future.

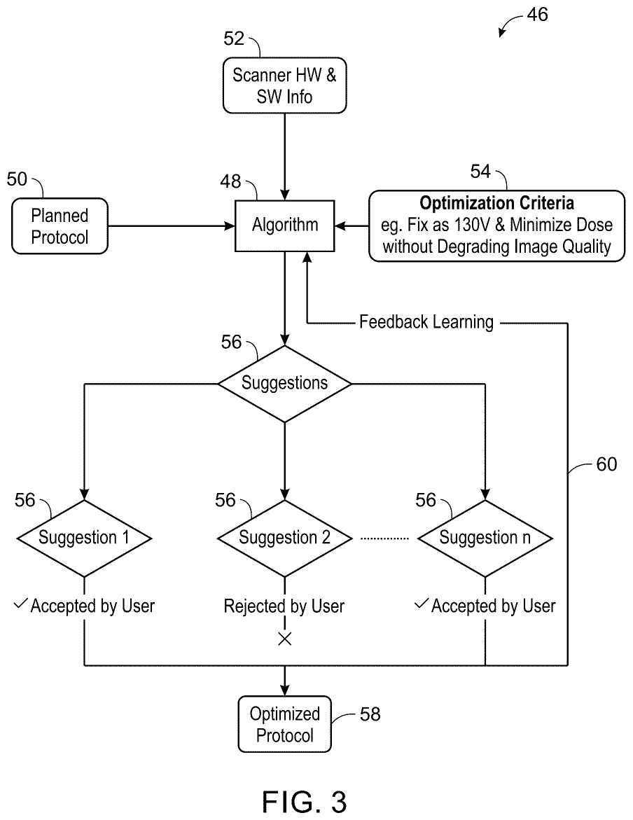

FIG. 3 is a flow chart of a process 46 for optimizing protocols for medical imaging scanners (e.g., individual protocol optimization). The process 46 includes inputting into an artificial intelligence-based algorithm 48 a planned protocol 50 (from the organization for a medical imaging scanner), information 52 specific to the hardware and software of the medical imaging scanner, and user input of desired optimization criteria 54. The artificial intelligence-based algorithm 48 outputs suggested protocol parameters 56 to optimize the planned protocol 50. The user, via interactive user interface, accepts and/or rejects the various suggested protocol parameters. Based on the suggested protocol parameters 56 that are accepted, an optimized protocol 58 for the planned protocol 50 is outputted. The optimized protocol is utilized to alter the settings of the medical imaging scanner when the optimized protocol is utilized for a scan of a subject with the medical imaging scanner. In addition, the suggested protocol parameters 56 that are accepted are utilized to provide feedback learning (e.g., RLHF) to the artificial intelligence-based algorithm 48 as indicated by reference numeral 60.

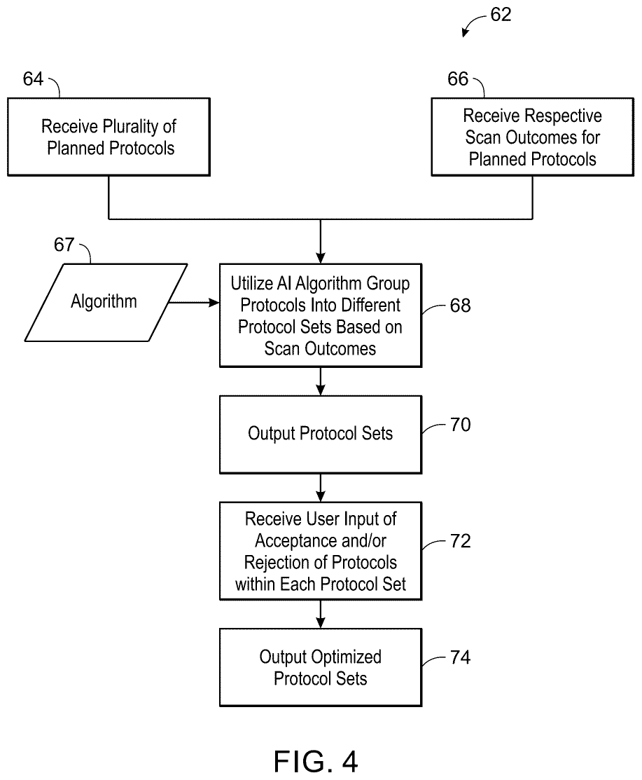

FIG. 4 is a flow chart of a method 62 for optimizing protocols for medical imaging scanners (e.g., protocol set optimization). One or more steps of the method 62 may be performed by one or more components of the protocol optimization device 12 in FIG. 1.

The method 62 includes receiving a plurality of planned protocols from an organization for the medical imaging scanner (block 64). The method 62 also includes receiving respective scan outcomes for each planned protocol of the plurality of planned protocols (block 66). The plurality of planned protocols produce duplicate scan outcomes. The method 62 further includes utilizing an artificial intelligence-based algorithm 67 to group the plurality of planned protocols into different protocol sets based on the respective scan outcomes (block 68). The method 62 even further includes outputting from the artificial intelligence-based algorithm 67 the different protocol sets (e.g., display on a display having an interactive user interface) (block 70). The method 62 yet further includes receiving user input of acceptance of one or more planned protocols within each protocol set of the different protocol sets and/or rejection of one or more of the planned protocols within each protocol set of the different protocol sets (e.g., via the interactive user interface) (block 72). The method 62 further includes outputting from the artificial intelligence-based algorithm 67 optimized protocol sets for the plurality of planned protocols based on the one or more planned protocols within each protocol set of the different protocol sets accepted via the user input (block 74). The outputted optimized protocol sets lack duplicate outcomes. The artificial intelligence-based algorithm 67 in the method 62 enables the review of all the planned protocols and identifies which protocols have the same outcome in terms of scanning to eliminate the lower performing protocols.

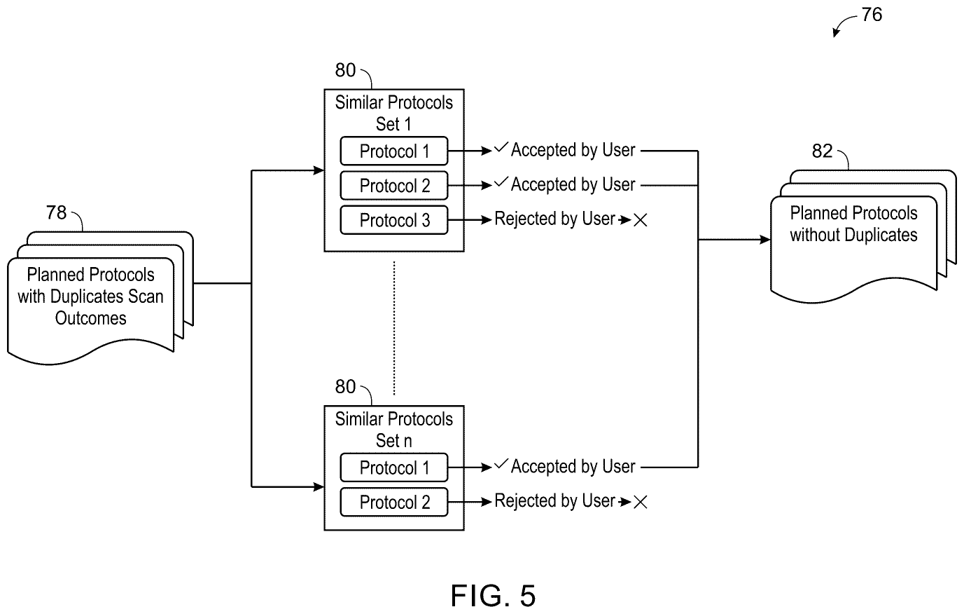

FIG. 5 is a flow chart of a process 76 for optimizing protocols for medical imaging scanners (e.g., protocol set optimization). The process 76 includes inputting planned protocols 78 (from an organization for a medical imaging scanner) along with respective scan outcomes (including duplicate scan outcomes) into artificial intelligence-based algorithm. The artificial intelligence-based algorithm groups the plurality of planned protocols 78 into different protocol sets 80 based on the respective scan outcomes and outputs the different protocol sets 80. The user, via interactive user interface, accepts and/or rejects planned protocols within each protocol set 80 of the different protocol sets 80. Based on the protocols that are accepted with each protocol set 80, optimized protocol sets 82 for the plurality of planned protocols are outputted that lack duplicate outcomes.

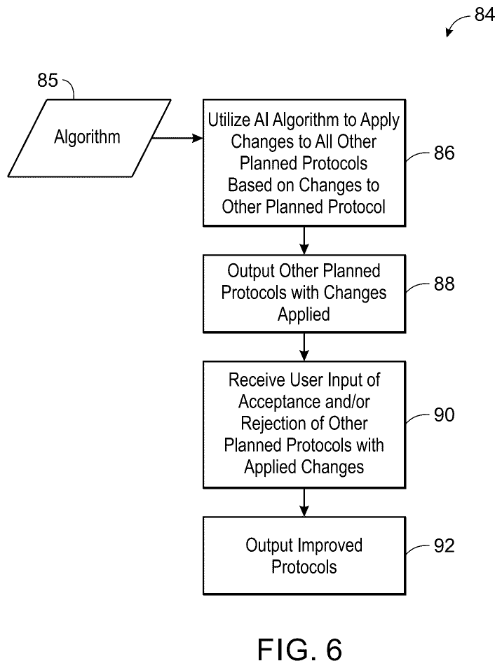

FIG. 6 is a flow chart of a method 84 for optimizing protocols for medical imaging scanners (e.g., via propagation). One or more steps of the method 84 may be performed by one or more components of the protocol optimization device 12 in FIG. 1. The method 84 enables the latest changes/optimizations to one planned protocol to be propagated to all the other planned protocols at once.

The method 84 includes utilizing an artificial intelligence-based algorithm 85 to apply changes/optimizations to all other planned protocols from an organization for a medical imaging scanner based on respective changes to a planned protocol to generate an optimized protocol (block 86). The method 84 also includes outputting from the artificial intelligence-based algorithm 85 the other planned protocols with applied changes (e.g., display on a display having an interactive user interface) (block 88). The method 84 further includes receiving user input of acceptance of one or more of the other planned protocols with the applied changes and/or rejection of one or more of the other planned protocols with the applied changes (e.g., via the interactive user interface) (block 90). The method 84 even further includes outputting from the artificial intelligence-based algorithm 85 respective improved/optimized protocols for the other planned protocols where the applied changes are accepted via the user input (block 92). It should be noted that any changes to other planned protocols may result in changes to the original optimized protocol that was utilized to change all of the other planned protocols. In addition, any changed/optimized protocol may be changed to a previously saved state via user request.

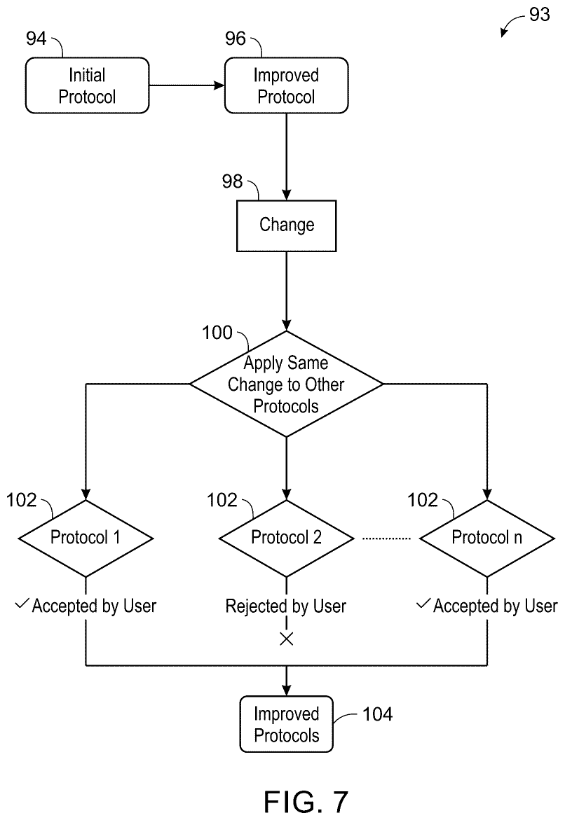

FIG. 7 is a flow chart of a process 93 for optimizing protocols for medical imaging scanners (e.g., via propagation). As depicted in FIG. 7, an initial planned protocol 94 is improved/optimized to generate an improved/optimized protocol 96 via one of the techniques disclosed in the present disclosure. An artificial intelligence-based algorithm is utilized to apply any changes/optimizations 98 made to the initial planned protocol 94 as indicated by reference numeral 100. The artificial intelligence-based algorithm outputs the other protocols 102 with the changes 98 applied to them. Based on the protocols 102 that are accepted with changes 98 applied, improved/optimized protocol 104 for other planned protocols are outputted. Any changes to the improved protocols 104 may be propagated to the improved protocol 96 as well.

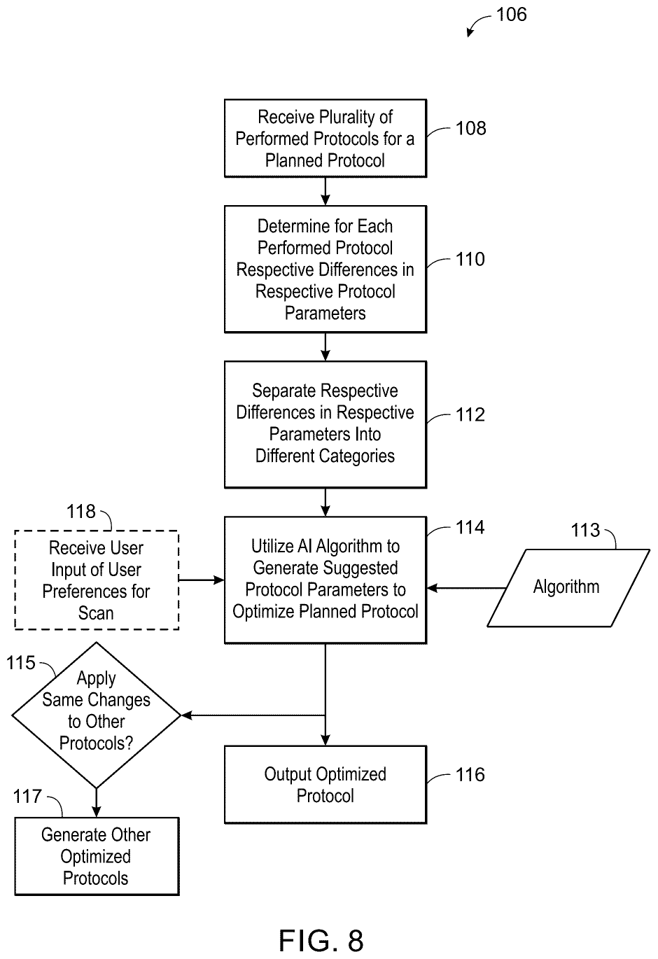

Often planned protocols are adjusted before performing the scan. These adjustments can vary from a trivial parameter that does not impact the image quality (e.g., Auto Voices) to dose sensitive fields such as kV, milliamperes, and so forth. A lot of a radiologist's time is wasted on adjusting these parameters for each scan across various protocols. These adjusted protocols which are actually used in scanning are performed protocols. For a single planned protocol, there can be many variations of performed protocols. FIGS. 8-11 relate to optimization of planned protocols (e.g. individually or as a set) based on past performed scans. FIG. 8 is a flow chart of a method 106 for optimizing protocols for medical imaging scanners (e.g., individual protocol optimization based on past performed scans). One or more steps of the method 106 may be performed by one or more components of the protocol optimization device 12 in FIG. 1.

The method 106 includes receiving a plurality of performed protocols for a planned protocol, wherein each performed protocol of the plurality of performed protocols was performed during a respective scan with respective protocol parameters (block 108). The method 106 also includes determining for each performed protocol of the plurality of performed protocols respective differences in the respective protocol parameters from protocol parameters of the planned protocol (block 110). The method 106 further includes separating the respective differences in the respective parameters into different categories (block 112). The method 106 even further includes utilizing an artificial intelligence-based algorithm 113 to generate suggested protocol parameters to optimize the planned protocol based at least on the plurality of performed protocols and the different categories (block 114). The method 106 further includes outputting from the artificial intelligence-based algorithm 113 an optimized protocol for the planned protocol based on the one or more of the suggested protocol parameters (block 116). In certain embodiments, the method 106 includes receiving user input of user preferences for the scan (block 118). An example of a user preference includes picking suggestions that lower the does without loss in image quality. In certain embodiments, the method 106 includes outputting from the artificial intelligence-based algorithm 113 the optimized protocol for the planned protocol based on the one or more of the suggested protocol parameters and user preferences (block 116). The optimized protocol is utilized to alter the settings of the medical imaging scanner when the optimized protocol is utilized for a scan of a subject with the medical imaging scanner. In certain embodiments, the method 106 includes applying the same changes to other planned protocols (as indicated by block 115) to generate other optimized protocols (block 117.

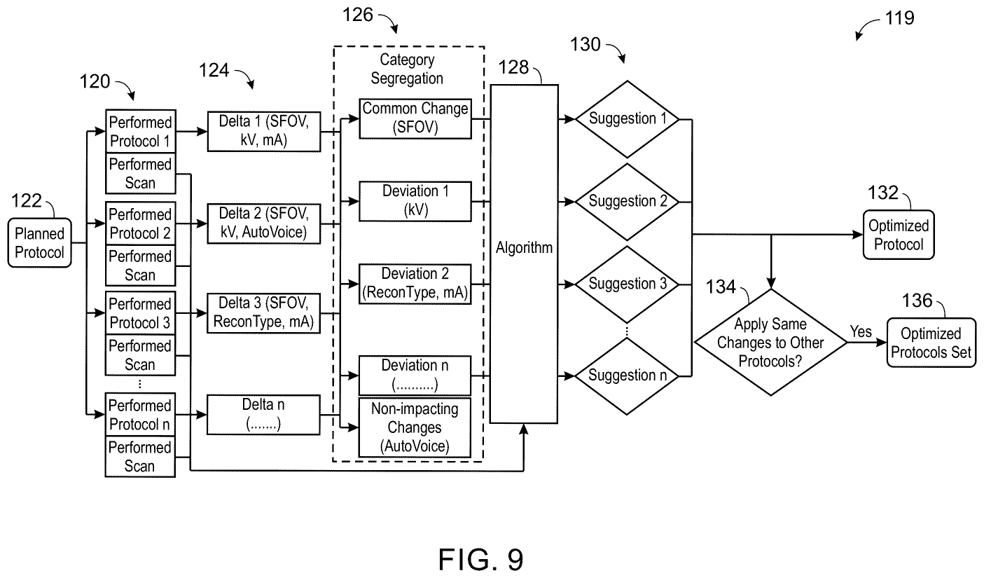

FIG. 9 is a flow chart of a process 119 for optimizing protocols for medical imaging scanners (e.g., individual protocol optimization based on past performed scans). As depicted in FIG. 9, the process 119 includes obtaining performed protocols 120 (i.e., performed scans) for a planned protocol 122 where the performed protocols 120 were performed during a respective scan with respective protocol parameters. For each performed protocol 120, the process 119 includes determining respective differences (as indicated by reference numeral 124) in the respective protocol parameters from protocol parameters of the planned protocol. The process 119 also includes separating the respective differences 124 in the respective parameters into different categories 126. The different categories 126 and the performed protocols 120 (i.e., DICOM images of the performed scans) are inputted into an artificial intelligence-based algorithm 128. The algorithm 128 may read the DICOM images to identify which protocols performed best. For example, changing mA from 110 to 125 improved quality but increasing from 110 to 140 mA produced similar results as 125 mA. Hence, the algorithm 128 will pick 125 mA in this scenario since dose will be lower in 125 mA compared to 140 mA. The artificial intelligence-based algorithm 128 is utilized to generate suggested protocol parameters 130 to optimize the planned protocol 122 based at least on the plurality of performed protocols 120 (i.e., performed scans) and the different categories 126. The artificial intelligence-based algorithm 128 outputs an optimized protocol 132 for the planned protocol 122. The optimized protocol 132 is utilized to alter the settings of the medical imaging scanner when the optimized protocol 132 is utilized for a scan of a subject with the medical imaging scanner. In certain embodiments, the process 119 includes applying the same changes to other planned protocols (as indicated by reference numeral 134) to generate an optimized protocol set 136.

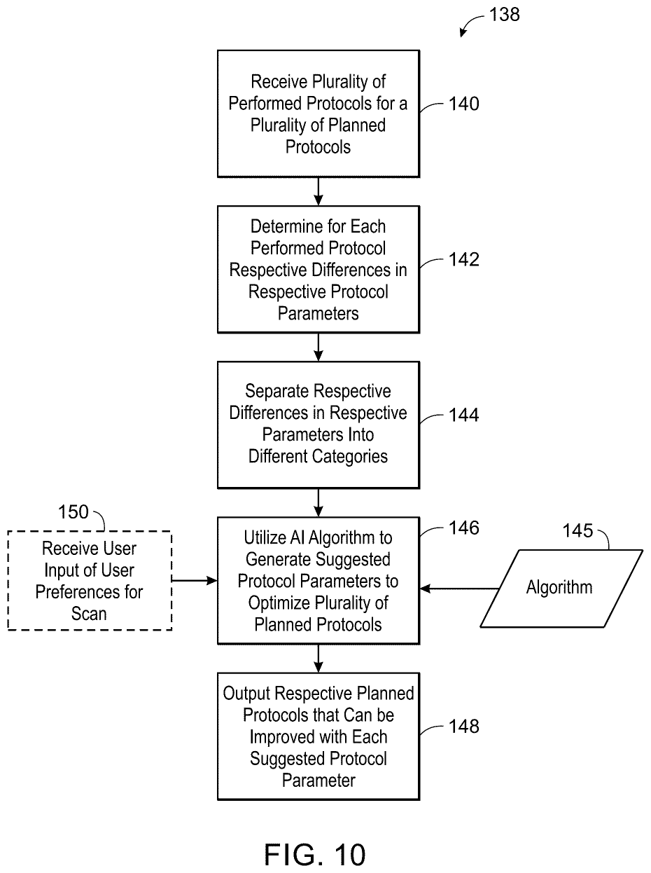

FIG. 10 is a flow chart of a method 138 for optimizing protocols for medical imaging scanners (e.g., protocol set optimization based on past performed scans). One or more steps of the method 138 may be performed by one or more components of the protocol optimization device 12 in FIG. 1. In certain cases, a particular optimization was never applied to performed versions of a particular planned protocol but the method 138 enables a particular optimization to be learned from performed versions of other planned protocols.

The method 138 includes receiving a plurality of performed protocols for a plurality of planned protocols from an organization for a medical imaging scanner, wherein each performed protocol of the plurality of performed protocols was performed during a respective scan with respective protocol parameters (block 140). The method 138 also includes determining for each performed protocol of the plurality of performed protocols for each respective planned protocol of the plurality of planned protocols respective differences in the respective protocol parameters from protocol parameters of the respective planned protocol (block 142). The method 138 further includes separating the respective differences in the respective parameters into different categories for the plurality of performed protocols for each respective planned protocol of the plurality of planned protocols (block 144). The method 138 even further includes utilizing an artificial intelligence-based algorithm 145 to generate suggested protocol parameters to optimize the plurality of planned protocols based at least on the plurality of performed protocols and the different categories for the plurality of planned protocols (block 146). The method 138 further includes outputting from the artificial intelligence-based algorithm 145 respective planned protocols that can be improved with each suggested protocol parameter of the suggested protocol parameters (block 148). In certain embodiments, the method 138 includes receiving user input of user preferences for the scan (block 150). An example of a user preference includes picking suggestions that lower the dose without loss in image quality. In certain embodiments, the method 138 includes utilizing the artificial intelligence-based algorithm 145 to generate/output suggested protocol parameters to optimize the plurality of planned protocols based on the user preferences, the plurality of performed protocols and the different categories for the plurality of planned protocols (block 146).

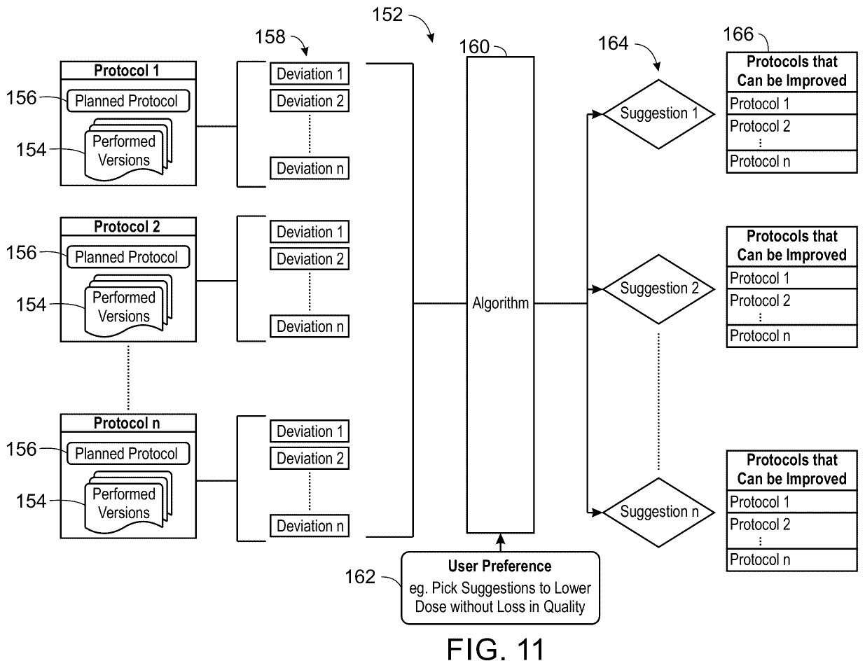

FIG. 11 is a flow chart of a process 152 for optimizing protocols for medical imaging scanners (e.g., protocol set optimization based on past performed scans). As depicted in FIG. 11, the process 152 includes obtaining performed protocols 154 for a plurality of planned protocols 156 where the performed protocols 154 were performed during a respective scan with respective protocol parameters. For each performed protocol 154, the process 152 includes determining respective differences (as indicated by reference numeral 158) in the respective protocol parameters from protocol parameters of the respective planned protocol. In certain embodiments, the process 152 also includes separating the respective differences 158 in the respective parameters into different categories. The respective differences 158 (or different categories) and, in certain embodiments, the performed protocols 154 for each planned protocol 156 are inputted into an artificial intelligence-based algorithm 160. In certain embodiments, the process 152 includes receiving user preferences for a scan as indicated by reference numeral 162. The artificial intelligence-based algorithm 160 is utilized to generate suggested protocol parameters 164 to optimize the plurality of planned protocols 156 based at least on the plurality of performed protocols 154 and the respective differences 158. The artificial intelligence-based algorithm 160 outputs respective planned protocols that can be improved (as indicated by reference numeral 166) with each suggested protocol parameter of the suggested protocol parameters.

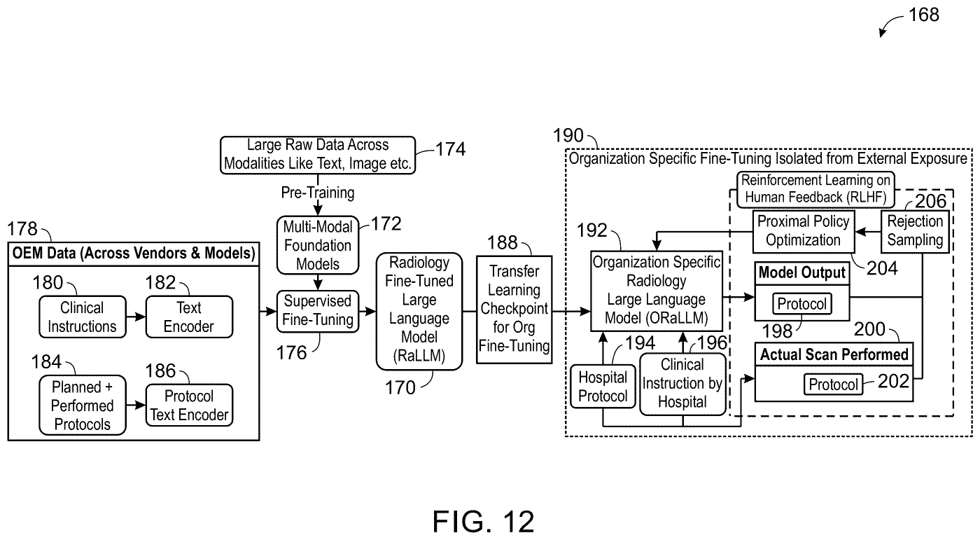

FIG. 12 is a flow chart of a process 168 for generating a generative artificial intelligence model for use in creating/optimizing protocols. The process 168 includes generating a radiology fine-tuned large language model (RaLLM) 170. Generating the radiology fine-tuned large language model 170, begins with the pre-training of multi-modal foundation models 172 (e.g., different open source models) with a large amount of data 174 across different modalities (e.g., text, image, etc.). The multi-modal foundation models 172 are then subjected to supervised fine-tuning 176 utilizing original equipment manufacturer (OEM) data 178 across different vendors and different models of medical imaging scanners. The OEM data 178 includes clinical instructions 180 which are processed by a text encoder 182. The OEM data 178 also includes planned and performed protocols 184 that are processed by a protocol text encoder 186.

The process 168 then includes transferring (as indicated by reference numeral 188) the radiology fine-tuned large language model 170 (e.g., to an organization) for organization specific fine-tuning 190. Organization specific fine-tuning based on the organization's planned protocols occurs without external exposure (e.g., to entities outside the organization). Organization specific fine-tuning 190 includes generating an organization specific radiology large language model (ORaLLM) 192. Generating the organization specific radiology large language model 192, includes fine-tuning utilizing hospital protocols 194 and clinical instructions 196 by the hospital. The organization specific radiology large language model 192 outputs protocols 198 (e.g., optimized or created protocols). These outputted protocols 198 and actual scans performed 200 and the associated protocols 202 with those scans 200 are utilized as feedback in further training or fine-tuning the organization specific radiology large language model 192. In particular, reinforcement learning on human feedback (RLHF) may be utilized. RLHF is a model training procedure that is applied to a fine-tuned language model to further align model behavior with human preferences and instruction following. Data is collected that represents human sampled human preferences, whereby human annotators selected which of two model outputs they prefer. This human feedback is subsequently used to train a reward model, which learns patterns in the preferences of the human annotators and can then automate preference decisions.

As depicted in FIG. 12, the process 168 includes utilizing proximal policy optimization (PPO) 204 based on rejection sampling 206 determined by the outputted protocols 198 (e.g., model protocols) and actual scans performed 200 and the associated protocols 202 with those scans 200. PPO operates on a policy gradient approach, where the agent directly learns a policy, typically parameterized by a neural network. The agent collects a set of trajectories under its current policy, and then updates the policy to maximize a specially designed objective function. This process is repeated iteratively, allowing the policy to gradually improve over time. An agent tries different actions and learns a policy that predicts which action to take in each state. The policy is updated based on the experiences, but instead of drastically changing the policy based on recent success or failure, PPO makes smaller, incremental changes. This way, the agent avoids drastically changing its strategy based on limited new information, leading to a more stable and consistent learning process. In the traditional model of optimizing human derived preferences via reinforcement learning, the typical method utilized has been to use an auxiliary reward model and fine-tune the model of interest so that it maximizes this given reward via the machinery of reinforcement learning. Intuitively, the reward model is utilized to provide feedback to the model that is be being optimized so that it generates high-reward samples more often and low-reward samples less often. At the same time, a frozen reference model is utilized to make sure that whatever is generated does not deviate too much and continues to maintain generation diversity.

In certain embodiments, direct policy optimization (DPO) may be utilized for fine-tuning. The DPO is prompted with instructions to generate a protocol (x) for the ORaLLM 192. Unlike traditional alignment techniques, which are based on reinforcement learning, DPO recasts the alignment formulation as a simple loss function that can be optimized directly on a dataset of preferences {(x, y_{w}, yₗ)}, where x is a prompt and y_{w}, yₗ are the preferred and nonpreferred response. The DPO formulation bypasses the reward modeling step and directly optimizes the language model on preference data via a key insight: namely an analytical mapping from the reward function to the optimal reinforcement learning policy that enables transforming reinforcement learning loss over the reward and reference models to a loss over the reference model directly. The mapping intuitively measures how well a given reward function aligns with the given preference data. DPO starts with the optimal solution to the RLHF loss and via a change of variables derives a loss over only the reference model.

The radiology fine-tuned large language model 170 trained on OEM data may not always produce satisfactory suggestions due to limitations in the training dataset compared to the vast variance of actual field data. To overcome this limitation, the radiology fine-tuned large language model 170 may be updated or further refined based on receiving user feedback from different organizations.

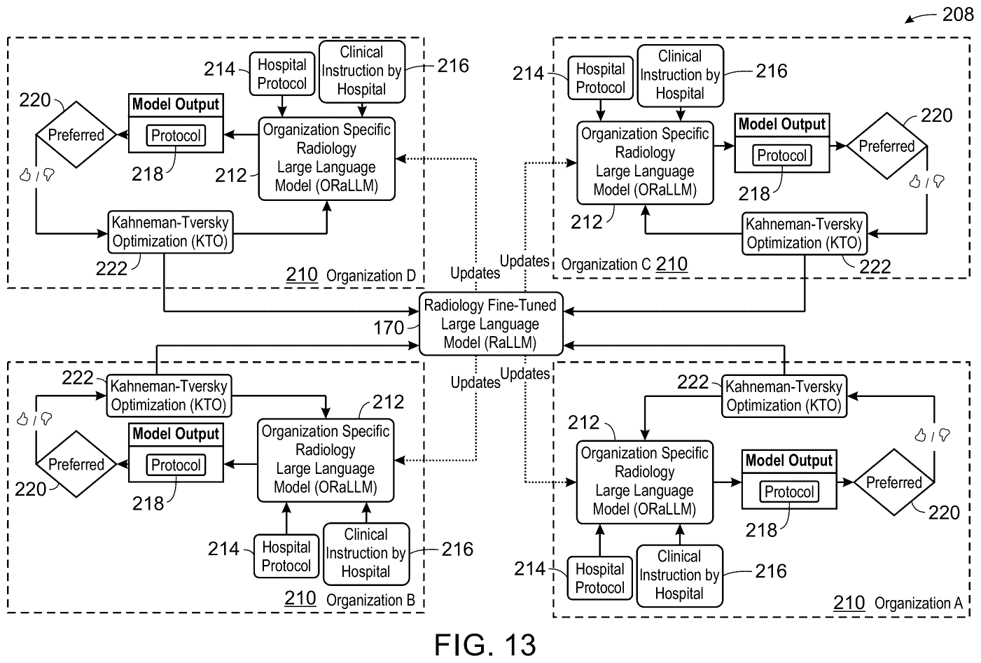

FIG. 13 is a flow chart of a process 208 for refining a generative artificial intelligence model (e.g., the radiology fine-tuned large language model 170) for use in in creating/optimizing protocols. The process 208 provides for the validation/improvement of the radiology fine-tuned large language model 170. Specifically, the radiology fine-tuned large language model 170 is subject to respective organization specific fine-tuning by different organizations 210 (e.g., organizations A, B, C, and D) to generate a respective organization specific radiology large language model 212. Generating the respective organization specific radiology large language model 212, includes fine-tuning utilizing hospital protocols 214 and clinical instructions 216 by the hospital for the respective organization. The organization specific radiology large language model 212 outputs a protocol 218 (e.g., optimized or created). User input as to whether the model generated protocol 218 is preferred or not is provided as indicated by reference numeral 220. Kahneman-Tversky optimization (KTO) 222 is utilized (base on the user input) to further refine the radiology fine-tuned large language model 170. This process occurs with each of the organizations 210. Updated versions of the radiology fine-tuned large language model 170 are then provided to the organizations 210.

Like most alignment methods, DPO (Direct Policy Optimization) requires a dataset of paired preferences (as noted above), where annotators label which response is better according to a set of criteria like helpfulness or harmfulness. In practice, creating these datasets is a time consuming and costly endeavor. However, with KTO, the loss function is entirely defined in terms of individual examples that have been labelled as good (thumbs up) or bad (thumbs down). These labels are much easier to acquire in practice.

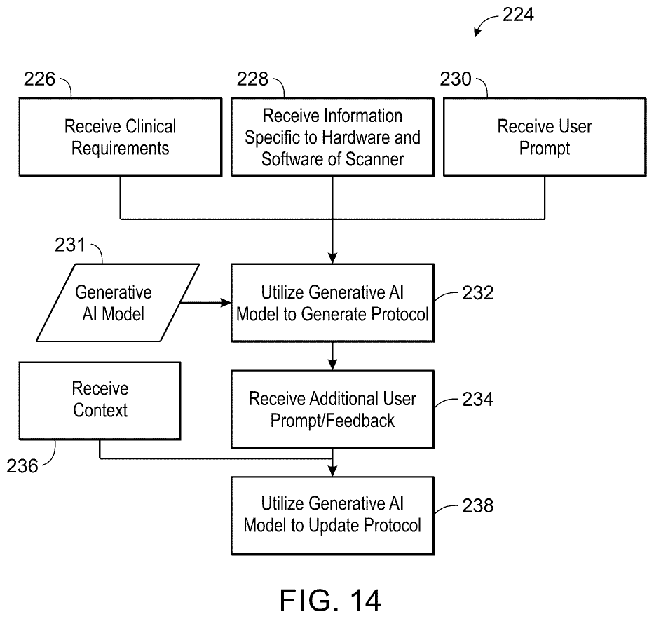

FIG. 14 is a flow chart of a method 224 for creating new protocols for medical imaging scanners. One or more steps of the method 224 may be performed by one or more components of the protocol optimization device 12 in FIG. 1.

The method 224 includes receiving clinical requirements (e.g., clinical instructions) for a scan using a medical imaging scanner of an organization (block 226). The method 224 also includes receiving information specific to hardware and software of the medical imaging scanner (block 228). The method 224 further includes receiving a user prompt for creating a protocol for the scan (block 230). The user prompt may include one or more desired outcomes for the scan. The method 224 even further includes utilizing a generative artificial intelligence-based model 231 (e.g., ORaLLM 192 in FIG. 12) to generate a protocol for performing the scan using the medical imaging scanner based on the clinical requirements, the information specific to hardware and software of the medical imaging scanner, and the one or more desired outcomes for the scan (block 232). In certain embodiments, the method 224 includes receiving additional user prompt that may include feedback (e.g., user input of additional one or more desired outcomes for the scan) (block 234). In certain embodiments, the method 224 also includes receiving context from the generative artificial intelligence-based model 231 (block 236). In certain embodiments, the method 224 further includes utilizing the generative artificial intelligence-based model 231 to update the protocol to generate an updated protocol (e.g., enhanced or improved protocol) based on the context and the additional user input (block 238).

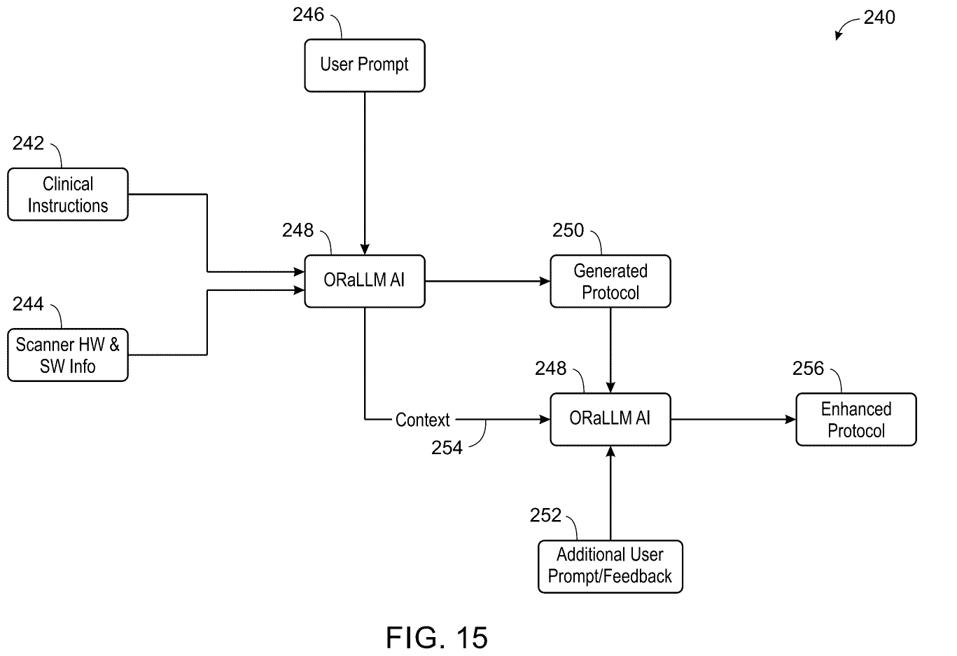

FIG. 15 is a flow chart of a process 240 for creating new protocols for medical imaging scanners. The process 240 includes inputting clinical requirements 242 (e.g., clinical instructions) for a scan using a medical imaging scanner of an organization, information 244 specific to the hardware and software of the medical imaging scanner, and user prompt 246 for creating a protocol for the scan that includes one or more desired outcomes for the scan into ORaLLM 248 (e.g., similar to ORaLLM 192 in FIG. 12). The ORaLLM 248 generates or creates a protocol 250 based on these inputs. The ORaLLM 248 may receive an additional user prompt/feedback 252 and context 254 from the ORaLLM 248 to generate an enhanced (e.g., improved) protocol 256 from the originally generated protocol 250.

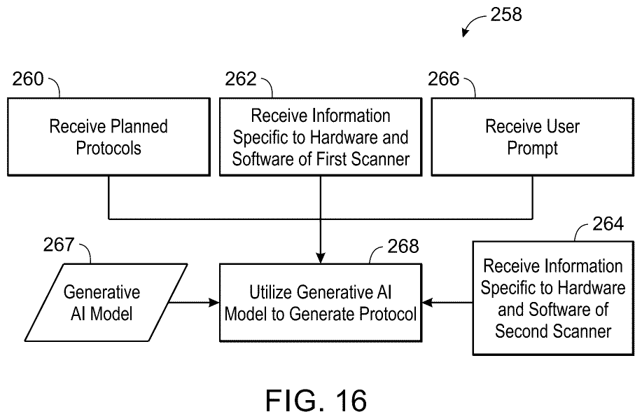

FIG. 16 is a flow chart of a method 258 for translating protocols for medical imaging scanners across scanner models and vendors. One or more steps of the method 258 may be performed by one or more components of the protocol optimization device 12 in FIG. 1.

The method 258 includes receiving planned protocols (e.g., existing planned protocols) from an organization for performing a scan with a first medical imaging scanner (block 260). The method 258 also includes receiving information specific to hardware and software of the first medical imaging scanner (block 262). The method 258 further includes receiving additional information specific to hardware and software of a second medical imaging scanner different from the first medical imaging scanner, wherein the second medical imaging scanner is of a different manufacturer and/or a different model from the first medical imaging scanner (block 264). The method 258 even further includes receiving a user prompt for translating existing planned protocols for the first medical imaging scanner to the second medical imaging scanner (block 266). The user prompt may include user input of one or more desired outcomes for a respective scan with the second medical imaging scanner. The method 258 still further includes utilizing a generative artificial intelligence-based model 267 (e.g., ORaLLM 192 in FIG. 12) to generate a protocol for performing the respective scan using the second imaging scanner based on the existing planned protocols and the one or more desired outcomes for the respective scan (block 268).

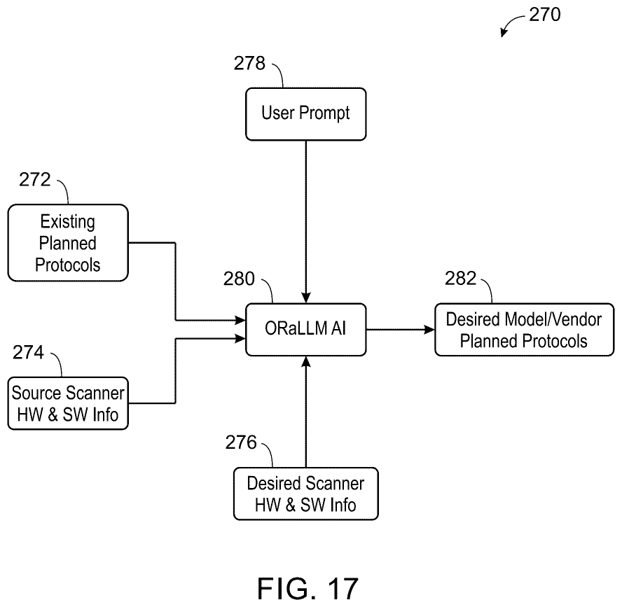

FIG. 17 is a flow chart of a process 270 for translating protocols for medical imaging scanners across scanner models and vendors. The process 270 includes inputting planned protocols 272 (e.g., existing planned protocols) from an organization for performing a scan with a first medical imaging scanner, information 274 specific to hardware and software of the first medical imaging scanner, additional information 276 specific to hardware and software of a second medical imaging scanner different from the first medical imaging scanner, and user prompt 278 for translating existing planned protocols for the first medical imaging scanner to the second medical imaging scanner (the user prompt may include user input of one or more desired scan outcomes for the second medical imaging scanner) into ORaLLM 280 (e.g., similar to ORaLLM 192 in FIG. 12). The ORaLLM 280 generates desired planned protocols 282 for the second medical imaging scanner based on these inputs (which may be across vendors and models).

Technical effects of the disclosed embodiments include enabling hospitals to improve efficiency and to obtain a better technical edge with their optimized planned protocol set. Technical effects of the disclosed embodiments include enabling hospitals to maintain fewer or smaller protocol sets as duplicated protocols yielding the same scan outcome are eliminated. Technical effects of the disclosed embodiments include enabling a reduction in processing time and/or memory requirements for creating new protocols (e.g., where the protocols are utilized to execute instructions with scanning devices and to obtain medical images) for hospitals using generative artificial intelligence. The newly created protocols (created via artificial intelligence) will require less manual tweaking since they were fine-tuned on an organization's own protocol set to match each organization's preferences and tastes. Technical effects of the disclosed embodiments include enabling hospitals to save time and money by translating their own optimized protocol set to a different scanner across models and vendors instead of creating it from scratch. Technical effects of the disclosed embodiments include providing better scan results for patients which results in a faster diagnosis due to optimized protocols by hospitals. Technical effects of the disclosed embodiments include enabling the same scan results for a patient for a given prescription across various vendor scanners.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

The disclosure also provides support for a computer-implemented method for optimizing protocols for medical imaging scanners, comprising: receiving, at a processing system comprising one or more processors, a planned protocol from an organization for a medical imaging scanner; utilizing, via the processing system, an artificial intelligence-based algorithm to generate suggested protocol parameters to optimize the planned protocol based at least on the planned protocol; outputting, via the processing system, from the artificial intelligence-based algorithm an optimized protocol for the planned protocol based on one or more of the suggested protocol parameters; modifying, via the processing system, settings of the medical imaging scanner when the optimized protocol is utilized for a scan of a subject with the medical imaging scanner; and executing, via the processing system, a scan of the subject with the medical imaging scanner utilizing the optimized protocol. In a first example of the method, the method further comprises: receiving, at the processing system, user input of desired optimization criteria; receiving, at the processing system, information specific to hardware and software of the medical imaging scanner; and utilizing, via the processing system, the artificial intelligence-based algorithm to generate suggested protocol parameters to optimize the planned protocol based on the planned protocol, the desired optimization criteria, and the information specific to the hardware and the software of the medical imaging scanner. In a second example of the method, optionally including the first example, the method further comprises: receiving, at the processing system, additional user input of acceptance of one or more of the suggested protocol parameters and/or rejection of one or more of the suggested protocol parameters; and outputting, via the processing system, from the artificial intelligence-based algorithm the optimized protocol for the planned protocol based on the one or more of the suggested protocol parameters accepted via the additional user input. In a third example of the method, optionally include one or both of the first and second examples, the method further comprising: training, via the processing system, the artificial intelligence-based algorithm based on the one or more of the suggested protocol parameters accepted via the additional user input. In a fourth example of the method, optionally including one or more or each of the first through third examples, the method further comprises: receiving, at the processing system, a plurality of planned protocols from the organization for the medical imaging scanner; receiving, at the processing system, respective scan outcomes for each planned protocol of the plurality of planned protocols; utilizing, via the processing system, the artificial intelligence-based algorithm to group the plurality of planned protocols into different protocol sets based on the respective scan outcomes; outputting, via the processing system, from the artificial intelligence-based algorithm the different protocol sets; receiving, via the processing system, user input of acceptance of one or more planned protocols within each protocol set of the different protocol sets and/or rejection of one or more of the planned protocols within each protocol set of the different protocol sets; and outputting, via the processing system, from the artificial intelligence-based algorithm optimized protocol sets for the plurality of planned protocol sets based on the one or more planned protocols within each protocol set of the different protocol sets accepted via the user input. In a fifth example, optionally including one or more or each of the first through fourth examples, the method comprises: utilizing, via the processing system, the artificial intelligence-based algorithm to apply changes to all other planned protocols from the organization for the medical imaging scanner based on respective changes to the planned protocol to generate the optimized protocol; outputting, via the processing system, from the artificial intelligence-based algorithm the other planned protocols with applied changes; receiving, via the processing system, user input of acceptance of one or more of the other planned protocols with the applied changes and/or rejection of one or more of the other planned protocols with the applied changes; and outputting, via the processing system, from the artificial intelligence-based algorithm respective improved protocols for the other planned protocols where the applied changes are accepted via the user input. In a sixth example, optionally including one or more or each of the first through fifth examples, the method further comprises: receiving, at the processing system, a plurality of performed protocols for the planned protocol, wherein each performed protocol of the plurality of performed protocols was performed during a respective scan with respective protocol parameters; determining, via the processing system, for each performed protocol of the plurality of performed protocols respective differences in the respective protocol parameters from protocol parameters of the planned protocol; separating, via the processing system, the respective differences in the respective parameters into different categories; and utilizing, via the processing system, the artificial intelligence-based algorithm to generate suggested protocol parameters to optimize the planned protocol based at least on the plurality of performed protocols and the different categories. In a seventh example, optionally including the one or more or each of the first through sixth examples, the method further comprises: receiving, at the processing system, a plurality of performed protocols for a plurality of planned protocols from the organization for the medical imaging scanner, wherein each performed protocol of the plurality of performed protocols was performed during a respective scan with respective protocol parameters; determining, via the processing system, for each performed protocol of the plurality of performed protocols for each respective planned protocol of the plurality of planned protocols respective differences in the respective protocol parameters from protocol parameters of the respective planned protocol; separating, via the processing system, the respective differences in the respective parameters into different categories for the plurality of performed protocols for each respective planned protocol of the plurality of planned protocols; utilizing, via the processing system, the artificial intelligence-based algorithm to generate suggested protocol parameters to optimize the plurality of planned protocols based at least on the plurality of performed protocols and the different categories for the plurality of planned protocols; and outputting, via the processing system, from the artificial intelligence-based algorithm respective planned protocols that can be improved with each suggested protocol parameter of the suggested protocol parameters. In an eighth example, optionally including the one or more or each of the first through the seventh examples, the method further comprises: receiving, at the processing system, user input of user preferences for the scan; and utilizing, via the processing system, the artificial intelligence-based algorithm to generate suggested protocol parameters to optimize the plurality of planned protocols based on the user preferences, the plurality of performed protocols and the different categories for the plurality of planned protocols.

The disclosure also provides support for a computer-implemented method for optimizing protocols for medical imaging scanners, comprising: receiving, at a processing system comprising one or more processors, clinical requirements for a scan using a medical imaging scanner of an organization; receiving, at the processing system, information specific to hardware and software of the medical imaging scanner; receiving, at the processing system, user input of one or more desired outcomes for the scan; and utilizing, via the processing system, a generative artificial intelligence-based model to generate a protocol for performing the scan using the medical imaging scanner based on the clinical requirements and the one or more desired outcomes for the scan. In a first example of the method, the method further comprises: receiving, at the processing system, additional user input additional one or more desired outcomes for the scan; receiving, at the processing system, context from the generative artificial intelligence-based model; and utilizing, via the processing system, the generative artificial intelligence-based model to update the protocol to generate an updated protocol based on the context and the additional user input. In a second example of the method, optionally including the first example, the generative artificial intelligence-based model comprises a radiology large language model specific to the organization. In a third example of the method, optionally including one or both of the first and second examples, the radiology large language model is fine-tuned based on protocols from the organization for the medical imaging scanner. In a fourth example of the method, optionally including one or more or each of the first through the third examples, organization specific fine-tuning of the radiology large language model is isolated from external exposure. In a fifth example of the method, optionally including one or more or each of the first through fourth examples, prior to the organization specific fine-tuning, the generative artificial intelligence-based model is pre-trained based on original equipment manufacturer data for different manufacturers and different models of medical imaging scanners similar to the medical imaging scanner and the original equipment manufacturer data for the medical imaging scanner.

The disclosure also provides support for a computer-implemented method for optimizing protocols for medical imaging scanners, comprising: receiving, at a processing system comprising one or more processors, existing planned protocols from an organization for performing a scan with a first medical imaging scanner; receiving, at the processing system, information specific to hardware and software of the first medical imaging scanner; receiving, at the processing system, additional information specific to hardware and software of a second medical imaging scanner different from the first medical imaging scanner, wherein the second medical imaging scanner is of a different manufacturer and/or a different model from the first medical imaging scanner; receiving, at the processing system, user input of one or more desired outcomes for a respective scan with the second medical imaging scanner; and utilizing, via the processing system, a generative artificial intelligence-based model to generate a protocol for performing the respective scan using the second imaging scanner based on the existing planned protocols and the one or more desired outcomes for the respective scan. In a first example of the method, the generative artificial intelligence-based model comprises a radiology large language model specific to the organization. In a second example of the method, optionally including the first example, the radiology large language model is fine-tuned based on protocols from the organization for the first medical imaging scanner. In a third example of the method, optionally including one or both of the first and second examples, organization specific fine-tuning of the radiology large language model is isolated from external exposure. In a fourth example of the method, optionally including one or more or each of the first through third examples, prior to the organization specific fine-tuning, the generative artificial intelligence-based model is pre-trained based on original equipment manufacturer data for different manufacturers and different models of medical imaging scanners similar to the first medical imaging scanner and the original equipment manufacturer data for the first medical imaging scanner.

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for optimizing protocols for medical imaging scanners, comprising:
receiving, at a processing system comprising one or more processors, a planned protocol from an organization for a medical imaging scanner;
utilizing, via the processing system, an artificial intelligence-based algorithm to generate suggested protocol parameters to optimize the planned protocol based at least on the planned protocol;
outputting, via the processing system, from the artificial intelligence-based algorithm an optimized protocol for the planned protocol based on one or more of the suggested protocol parameters;
modifying, via the processing system, settings of the medical imaging scanner when the optimized protocol is utilized for a scan of a subject with the medical imaging scanner; and
executing, via the processing system, a scan of the subject with the medical imaging scanner utilizing the optimized protocol.

2. The computer-implemented method of claim 1, further comprising:
receiving, at the processing system, user input of desired optimization criteria;
receiving, at the processing system, information specific to hardware and software of the medical imaging scanner; and
utilizing, via the processing system, the artificial intelligence-based algorithm to generate suggested protocol parameters to optimize the planned protocol based on the planned protocol, the desired optimization criteria, and the information specific to the hardware and the software of the medical imaging scanner.

3. The computer-implemented method of claim 2, further comprising:
receiving, at the processing system, additional user input of acceptance of one or more of the suggested protocol parameters and/or rejection of one or more of the suggested protocol parameters; and
outputting, via the processing system, from the artificial intelligence-based algorithm the optimized protocol for the planned protocol based on the one or more of the suggested protocol parameters accepted via the additional user input.

4. The computer-implemented method of claim 3, further comprising training, via the processing system, the artificial intelligence-based algorithm based on the one or more of the suggested protocol parameters accepted via the additional user input.

5. The computer-implemented method of claim 1, further comprising:
receiving, at the processing system, a plurality of planned protocols from the organization for the medical imaging scanner;
receiving, at the processing system, respective scan outcomes for each planned protocol of the plurality of planned protocols;
utilizing, via the processing system, the artificial intelligence-based algorithm to group the plurality of planned protocols into different protocol sets based on the respective scan outcomes;
outputting, via the processing system, from the artificial intelligence-based algorithm the different protocol sets;
receiving, via the processing system, user input of acceptance of one or more planned protocols within each protocol set of the different protocol sets and/or rejection of one or more of the planned protocols within each protocol set of the different protocol sets; and
outputting, via the processing system, from the artificial intelligence-based algorithm optimized protocol sets for the plurality of planned protocols based on the one or more planned protocols within each protocol set of the different protocol sets accepted via the user input.

6. The computer-implemented method of claim 1, further comprising:
utilizing, via the processing system, the artificial intelligence-based algorithm to apply changes to all other planned protocols from the organization for the medical imaging scanner based on respective changes to the planned protocol to generate the optimized protocol;
outputting, via the processing system, from the artificial intelligence-based algorithm the other planned protocols with applied changes;
receiving, via the processing system, user input of acceptance of one or more of the other planned protocols with the applied changes and/or rejection of one or more of the other planned protocols with the applied changes; and
outputting, via the processing system, from the artificial intelligence-based algorithm respective improved protocols for the other planned protocols where the applied changes are accepted via the user input.

7. The computer-implemented method of claim 1, further comprising:
receiving, at the processing system, a plurality of performed protocols for the planned protocol, wherein each performed protocol of the plurality of performed protocols was performed during a respective scan with respective protocol parameters;
determining, via the processing system, for each performed protocol of the plurality of performed protocols respective differences in the respective protocol parameters from protocol parameters of the planned protocol;
separating, via the processing system, the respective differences in the respective protocol parameters into different categories; and
utilizing, via the processing system, the artificial intelligence-based algorithm to generate suggested protocol parameters to optimize the planned protocol based at least on the plurality of performed protocols and the different categories.

8. The computer-implemented method of claim 1, further comprising:
receiving, at the processing system, a plurality of performed protocols for a plurality of planned protocols from the organization for the medical imaging scanner, wherein each performed protocol of the plurality of performed protocols was performed during a respective scan with respective protocol parameters;
determining, via the processing system, for each performed protocol of the plurality of performed protocols for each respective planned protocol of the plurality of planned protocols respective differences in the respective protocol parameters from protocol parameters of the respective planned protocols;
separating, via the processing system, the respective differences in the respective parameters into different categories for the plurality of performed protocols for each respective planned protocol of the plurality of planned protocols;
utilizing, via the processing system, the artificial intelligence-based algorithm to generate suggested protocol parameters to optimize the plurality of planned protocols based at least on the plurality of performed protocols and the different categories for the plurality of planned protocols; and
outputting, via the processing system, from the artificial intelligence-based algorithm respective planned protocols that can be improved with each suggested protocol parameter of the suggested protocol parameters.

9. The computer-implemented method of claim 8, further comprising:
receiving, at the processing system, user input of user preferences for the respective scan; and
utilizing, via the processing system, the artificial intelligence-based algorithm to generate suggested protocol parameters to optimize the plurality of planned protocols based on the user preferences, the plurality of performed protocols and the different categories for the plurality of planned protocols.

10. A computer-implemented method for optimizing protocols for medical imaging scanners, comprising:
receiving, at a processing system comprising one or more processors, clinical requirements for a scan using a medical imaging scanner of an organization;
receiving, at the processing system, information specific to hardware and software of the medical imaging scanner;
receiving, at the processing system, user input of one or more desired outcomes for the scan; and
utilizing, via the processing system, a generative artificial intelligence-based model to generate a protocol for performing the scan using the medical imaging scanner based on the clinical requirements and the one or more desired outcomes for the scan.

11. The computer-implemented method of claim 10, further comprising:
receiving, at the processing system, additional user input additional one or more desired outcomes for the scan;
receiving, at the processing system, context from the generative artificial intelligence-based model; and
utilizing, via the processing system, the generative artificial intelligence-based model to update the protocol to generate an updated protocol based on the context and the additional user input.

12. The computer-implemented method of claim 10, wherein the generative artificial intelligence-based model comprises a radiology large language model specific to the organization.

13. The computer-implemented method of claim 12, wherein the radiology large language model is fine-tuned based on protocols from the organization for the medical imaging scanner.

14. The computer-implemented method of claim 13, wherein organization specific fine-tuning of the radiology large language model is isolated from external exposure.

15. The computer-implemented method of claim 14, wherein, prior to the organization specific fine-tuning, the generative artificial intelligence-based model is pre-trained based on original equipment manufacturer data for different manufacturers and different models of medical imaging scanners similar to the medical imaging scanner and the original equipment manufacturer data for the medical imaging scanner.

16. A computer-implemented method for optimizing protocols for medical imaging scanners, comprising:
receiving, at a processing system comprising one or more processors, existing planned protocols from an organization for performing a scan with a first medical imaging scanner;
receiving, at the processing system, information specific to hardware and software of the first medical imaging scanner;
receiving, at the processing system, additional information specific to hardware and software of a second medical imaging scanner different from the first medical imaging scanner, wherein the second medical imaging scanner is of a different manufacturer and/or a different model from the first medical imaging scanner;
receiving, at the processing system, user input of one or more desired outcomes for a respective scan with the second medical imaging scanner; and
utilizing, via the processing system, a generative artificial intelligence-based model to generate a protocol for performing the respective scan using the second medical imaging scanner based on the existing planned protocols and the one or more desired outcomes for the respective scan.

17. The computer-implemented method of claim 16, wherein the generative artificial intelligence-based model comprises a radiology large language model specific to the organization.

18. The computer-implemented method of claim 17, wherein the radiology large language model is fine-tuned based on protocols from the organization for the first medical imaging scanner.

19. The computer-implemented method of claim 18, wherein organization specific fine-tuning of the radiology large language model is isolated from external exposure.

20. The computer-implemented method of claim 19, wherein, prior to the organization specific fine-tuning, the generative artificial intelligence-based model is pre-trained based on original equipment manufacturer data for different manufacturers and different models of medical imaging scanners similar to the first medical imaging scanner and the original equipment manufacturer data for the first medical imaging scanner.
